(19) United States
(12) Patent Application Publication
(10) Pub. No.: US 2025/0018222 A1
(43) Pub. Date: Jan. 16, 2025

(54) DEVICE FOR LOADING AND PUSHING RADIOACTIVE SEEDS

(71) Applicants: UNIVERSITE DE LILLE, LILLE (FR); CENTRALE LILLE INSTITUT, VILLENEUVE D'ASCQ (FR); CENTRE NATIONAL DE LA RECHERCHE SCIENTIFIQUE, PARIS (FR)

(72) Inventors: Rochdi MERZOUKI, VILLENEUVE D'ASCQ (FR); Abdelkader BELAROUCI, AUBY (FR); Vincent COELEN, LA MADELEINE (FR); Fabien VERBRUGGHE, WALLERS (FR); Mario SANS LOPEZ, LILLE (FR)

(73) Assignees: UNIVERSITE DE LILLE, LILLE (FR); CENTRALE LILLE INSTITUT, VILLENEUVE D'ASCQ (FR); CENTRE NATIONAL DE LA RECHERCHE SCIENTIFIQUE, PARIS (FR)

(21) Appl. No.: 18/715,351

(22) PCT Filed: Dec. 1, 2022
(86) PCT No.: PCT/FR2022/052212

§ 371 Date: May 31, 2024

(30) Foreign Application Priority Data
Dec. 1, 2021 (FR) 2112809

(51) Int. Cl. A61N 5/10

(52) U.S. Cl. CPC A61N 5/1007; A61N 5/1027; A61N 2005/1009; A61N 2005/101; A61N 2005/1024

(57) ABSTRACT

A radioactive seed loading and pushing device suitable for a brachytherapy treatment, the device including: a seed reservoir containing radioactive seeds, free with respect to one another; a flexible push cable and a motor-driven device for driving and guiding the push cable configured to make the push cable switch from a retracted position, clearing a loading area for a radioactive seed, up to a deployed position ensuring pushing of the seed; and a system ensuring loading of a seed from the reservoir up to the loading area.

### FIELD

The present disclosure relates to a device for loading and pushing radioactive seeds, as well as to a brachytherapy treatment apparatus comprising a device for loading and pushing radioactive seeds and a flexible catheter which extends up to a hollow needle.

Such a treatment apparatus finds a particular application for treatment by brachytherapy with guidance under assistance by magnetic resonance imaging (MRI), and for example treatment by brachytherapy of the prostate.

Also, the present disclosure also relates to a brachytherapy equipment by magnetic resonance imaging guidance comprising an imaging scanner comprising a magnet and a magnet tunnel, as well as a brachytherapy apparatus for which the needle is positioned internally to the tunnel of the magnet; while the seed loading and pushing device according to the present disclosure is external to said tunnel of the magnet.

### BACKGROUND

The present disclosure relates to the field of devices used in radioactivity-based therapies. More particularly, the present disclosure relates to a device for loading and pushing radioactive seeds.

In general, the devices involved in the therapies based on the use of radioactivity should comply with some constraints, in particular when they are used in combination with imaging systems such as MRI, scanners or echography used to ensure guidance of the treatment. For example, besides the fact that they should be compatible with the used imaging system, they should also demonstrate some reliability in use thereof. Indeed, a reliability problem could result in damaging delays in the treatment protocol of a patient, or in delaying the number of patients who can be treated for example.

One of these therapies, so-called Brachytherapy, is an internal radiotherapy technique wherein the irradiation source is directly introduced into the tumour or immediately proximate thereto by means of a catheter. Several sources of irradiations are generally placed, and they may consist of one or more radioactive seed(s), for example iodine 125, Palladium Pd103, intraprostatic fiducial or positioning markers.

According to one example, a device for loading radioactive seeds from a reservoir of free seeds is known from the document U.S. Pat. No. 7,578,781 B2. The radioactive seeds are released, one-by-one, in a downstream branch splitting at its proximal end into two upstream branches. The first upstream branch comprises a pointed cable configured to perforate the tissues up to the area to be treated while blocking the possible release of a radioactive seed in the downstream branch. The second upstream branch comprises a cable allowing pushing the radioactive seed released in the downstream branch after removal of the pointed cable.

A major drawback of this loading type is its lack of reliability. In particular and according to the findings and observations specific to the Inventors, It has been found that the cable allowing pushing the released radioactive seed could be easily blocked with regards to the configuration of this device type, and in particular be stuck at the outlet of the seed reservoir.

The document U.S. Pat. No. 7,118,523 B2 is also known, which criticises the loading devices of the prior art based on a reservoir of free seeds, in that they are at the origin of reliability problems. According to the critics of U.S. Pat. No. 7,118,523 B2, this lack of reliability would result from the fact that the seeds are free with respect to one another in the cartridge, which would create a risk of clogging.

To address this lack, U.S. Pat. No. 7,118,523 B2 proposes a loading and pushing device using:
a coil on which a radioactive chain is wound, composed of radioactive seeds and (non-radioactive) spacers between the seeds, in alternation over the length of the chain, ensuring determined spacings between the consecutive seeds of the radioactive chain,
a pushing system comprising a motor-driven push cable, the push cable (then retracted) and the distal end of the chain then initially respectively arranged in two upstream guide branches, the two upstream branches meeting after junction thereof into a downstream branch forming a loading area,
a cutting system, arranged at the upstream branch ensuring guidance of the seed chain

Such a loading and pushing device operates as follows: the chain is actuated, unwound over a portion of length corresponding to the number of seeds to be inserted, at the cutting system, then the portion of length is sectioned from the rest of the radioactive chain by the cutting system

The sectioned seed chain is pushed by unwinding of the coil of seeds up to the downstream branch, then the radioactive chain (not sectioned) is wound so as to clear the junction leaving the sectioned chain of seeds in the loading area. The push cable is then deployed so as to push the portion of the chain of seeds, from the loading area up to the treatment area.

Such a device allows inserting into the area to be treated several radioactive seeds of the chain of radioactive seeds spaced apart from one another by bio-absorbable spacers.

Such a loading and pushing device has some disadvantages related in particular to the use of a chain of seeds that is more difficult to supply than free seeds. Furthermore, the spacers physically fix the spacing between the seeds, which generates a lack of flexibility of the device when other spacings are desired for the treatment.

A brachytherapy needle loading device is also known from the document US2004/0162458 A1 from a first radioactive seed cartridge, and a second spacer cartridge which are both in communication with a pre-loading chamber of the device.

Such a device is configured to load the needle of a train formed of radioactive seeds and spacers between the seeds when the needle is rigidly connected to a connection of the loading device. The hollow needle needs to be removed from the loading device, before use thereof for surgical deposits, namely seeds and spacers in the tissues of the prostate of the patient, which operations could be performed either manually by a practitioner, or by automated means. In any case, these automated means are distinct from the pre-loading device which is not configured for this operation.

### SUMMARY

The present disclosure improves the situation.

Thus, a radioactive seed loading and pushing device suitable for a brachytherapy treatment is proposed, the device comprising:
a reservoir of seeds containing radioactive seeds, free with respect to one another,
a flexible push cable, and a motor-driven guide and drive device of the push cable configured to make the push cable switch from a retracted position clearing a loading area for a radioactive seed into a deployed position ensuring pushing of the seed from the loading area up to a downstream treatment area, the drive and guide device comprising a first actuator and a guide system for the push cable comprising a downstream branch forming the loading area for the radioactive seed and an upstream branch, extending the downstream branch at a proximal end of the downstream branch, inside which upstream branch a push end of the push cable is configured to retract, in a retracted position of the push cable,
a system ensuring loading of a seed from the reservoir up to the loading area.

According to the present disclosure, the upstream branch is a first upstream branch, the system ensuring the loading of a seed from the seed reservoir up to the loading area comprising:
a second upstream branch, the downstream branch extending at its proximal end by splitting into said first upstream branch and said second upstream branch and wherein the seed reservoir opens into the second upstream branch, at a pre-loading area of a seed,
an actuation device comprising a second actuator and a push member configured to switch from a retracted initial position, beyond the proximal end, clearing the pre-loading area by enabling exit of a seed from the seed reservoir into the second branch up to a deployed position for which said push member moves the radioactive seed from the pre-loading area up to the loading area while preventing exit of a seed from the seed reservoir in the pre-loading area,
a control unit configured, starting from the retracted position of the push cable and the retracted initial position of the push member, to:
/a/advance said push member by the second actuator so as to push the radioactive seed from the pre-loading area into the second upstream branch up to the loading area in the downstream branch,
/b/retract said push member into the second upstream branch by the second actuator so as to clear the proximal end of the downstream branch,
/c/advance the push cable by the first actuator so as to push the radioactive seed from the loading area up to a treatment area.

Advantageously, such arrangements limit the risk of interference or jamming of the push cable, thereby guaranteeing a high reliability of the loading and pushing device. This increase in reliability guarantees the treatment schedule of a patient as well as the number of patients to be treated.

By treatment area, it may be understood an area comprising the tissues (for example, biological tissues) to be treated of the patient.

In one or more embodiment(s), the radioactive seed loading and pushing device is fixed, while the hollow needle can be moved, for example movable by means of a robot, thanks to the flexible catheter which allows for a freedom of movement between the movable hollow needle and the fixed radioactive seed loading and pushing device.

In one or more embodiment(s), the seed reservoir is a removable cartridge, and wherein the second branch comprises at the reloading area, a lateral opening (in particular an upper opening) as well as a support configured to ensure fastening of the cartridge, in a removable manner, at the lateral opening.

Thus, advantageously, the loading and pushing device can be supplied with radioactive seeds in a simply, rapidly and effectively, requiring no disassembly of the device and specific adjustments.

In one or more embodiment(s), the push member is a rectilinear rod, and wherein:
the downstream branch and the second upstream branch extend rectilinearly,
the downstream branch and the first upstream branch form a bend therebetween.

In one or more embodiment(s), the system ensuring loading of a seed from the seed reservoir up to the loading area comprises a detector targeting the loading area configured to detect a presence in the loading area, the detector emitting a signal at the input of the control unit, and wherein, in/a/, the control unit is configured to ensure the movement of the seed by the push member from the pre-loading area up to the detection of the seed by the detector in the loading area.

Thus, advantageously, the detector allows detecting the presence of the seed in the loading area before activating the push member.

In one or more embodiment(s), the control unit is configured to determine the initial position of the push member, at least in an initialisation mode, by:
/d/controlling the second actuator so as to move the push member until detection of the push member by the detector in the loading area,
/e/obtaining an actual position of the push member in the loading area,
/f/determining said retracted initial position of the push member on the basis of the obtained actual position.

Thus, advantageously, the three control steps allow accurately determining the position of the end of the push member.

In one or more embodiment(s), the second actuator comprises a motor, as well as a transmission comprising a gear comprising a gear wheel driven by the motor, and a rack secured to the push member.

In one or more embodiment(s), a cable detector targets a detection area in said first branch and wherein the control unit is configured to determine the retracted position of the push cable, at least in an initialisation mode, for which the first actuator moves the push cable until detection of the push end by the cable detector, namely that the cable is advanced if the latter is not initially detected, or on the contrary retracted if it is initially detected.

Thus, advantageously, it is possible to guarantee management of the intersection between the two branches to avoid collisions between the seed loading action and the seed deposition. The procedure also allows ensuring that the branch is free before launching loading of the seed or launching the deposition of the seed.

In one or more embodiment(s), the first actuator comprises a pair comprising a motor-driven roller and a pressing roller between which the push cable is pinched.

In one or more embodiment(s), a second pair comprises a roller coupled to a measurement means such as an incremental encoder and a second pressing roller between which the push cable is pinched.

Thus, advantageously, it may be possible to know the unwound length of the push cable at any time.

In one or more embodiment(s), a system for adjusting the pressure of the first and second pressing rollers, comprising a first support for the first pressing roller and a second support for the second pressing roller articulated to each other about an articulation axis, as well as a screw tightening system connecting the first support and the second support.

In one or more embodiment(s), the loading device comprises, upstream of the first upstream branch, a system for accumulating the push cable in the retracted position comprising a rigid sheath for the push cable, the sheath being shaped according to a helicoid.

Thus, advantageously, it is possible to store the push cable in a compact manner and reduce the overall size of the loading and pushing device.

According to another aspect, a method for loading and pushing a radioactive seed implemented by the control unit of the device is proposed, said method comprising:
/a/advancing a push member by the second actuator so as to push the radioactive seed from the pre-loading area in the second upstream branch up to the loading area in the downstream branch,
/b/retracting the push member in the second upstream branch by the second actuator so as to clear the proximal end of the downstream branch,
/c/advancing the push cable by the first actuator so as to push the radioactive seed from the loading area up to a treatment area.

A computer program comprising program code instructions for executing the steps of the method of the loading and pushing device according to the present disclosure when said program is executed on a computer.

According to another aspect, a brachytherapy apparatus is proposed comprising a radioactive seed loading and pushing device according to the present disclosure and a flexible catheter which extends up to a hollow needle, the catheter extending a distal end of the downstream branch of the loading device and wherein the push cable is configured to push the radioactive seed from the loading area up to a tip of the hollow needle.

By hollow needle (or needle), it could be understood a rigid hollow needle or a controllable hollow needle such as that one described in the document WO2015/153174, for example.

According to one embodiment, the apparatus comprises a radioactive head removably coupled to the end of the push cable by a fastening means, the cable being configured to be deployed by the first actuator until the radioactive end protrudes from the tip of the hollow needle.

In one or more embodiment(s), the brachytherapy treatment apparatus further comprises a guide assistance configured to guide the insertion of the hollow needle into the area to be treated and to guide the deposition of at least one seed, said guide assistance comprising a guide robot configured to be coupled with an imaging device, said guide robot being configured to be placed within or proximate to the imaging device and being further configured to move the hollow needle, and insert the latter into the area to be treated, said imaging device being configured to visualize the movement and the insertion of the hollow needle into the area to be treated, as well as the deposition of the radioactive seed.

In one or more embodiment(s), the guide robot may be further configured to be compatible with said imaging device when said imaging device uses magnetic field values comprised between 0 and 5 Teslas.

According to another aspect, a brachytherapy equipment by magnetic resonance imaging guidance is provided comprising an imaging scanner comprising a magnet and a magnet tunnel; as well as a brachytherapy apparatus according to the present disclosure for which the needle is positioned internally to the tunnel of the magnet; while the seed loading and pushing device is external to said tunnel of the magnet.

According to another aspect, a method is proposed for depositing at least one radioactive seed in a treatment area of a patient to be treated using a brachytherapy treatment apparatus according to the present disclosure or using a brachytherapy treatment equipment according to the present disclosure, said patient being installed within said imaging device and said deposition method being imaged in real-time by the imaging device which operates continuously, said method comprising:
/a1/inserting said hollow needle into the tissues of the patient comprised in the treatment area under control of the imaging device,
/b1/loading said at least one radioactive seed into the loading area,
/c1/pushing said at least one radioactive seed from the loading area up to said at least one tip of the hollow needle in order to deposit said at least one seed in line with the tip in the tissues of the patient under control of the imaging device.

By imaging, it could be understood the visualisation or the observation of the deposition method via the imaging device. The visualisation may correspond to the visualisation of a video stream comprising a set of images obtained by the imaging device.

In one or more embodiment(s), loading of the at least one radioactive seed may also be performed before insertion of the hollow needle into the tissues of the patient.

### BRIEF DESCRIPTION OF THE DRAWINGS

Other features, details and advantages will appear upon reading the detailed description hereinafter, and on analysis of the appended drawings, wherein:

FIG. 1 schematically illustrates a loading and pushing device in one ore more embodiment(s).

FIG. 2a shows a partial sectional view of the device 100 according to the plane XY.

FIG. 2b shows a partial sectional view of the device 100 according to the plane XZ.

FIG. 3a, FIG. 3b, FIG. 3c, FIG. 3d, and FIG. 3e schematically illustrate the method for loading a radioactive seed in the loading area of the device 100.

FIG. 4 illustrates a device for implementing the method of the present disclosure.

FIG. 5 is a sectional view of an apparatus according to the present disclosure comprising a loading and pushing device, a catheter and a hollow needle, the downstream branch of the device extended by the flexible catheter, which extends up to the hollow needle, enabling guidance of the needle in the patient by magnetic resonance imaging (MRI), the hollow needle positioned in a magnet tunnel of the magnet of the MRI scanner while the loading and pushing device; illustrated in FIG. 1, is external to the tunnel.

### DETAILED DESCRIPTION

The drawings and the description hereinafter essentially contain elements of a certain nature. Hence, they could not only be used to better understand the present disclosure, but also contribute to the definition thereof, where appropriate.

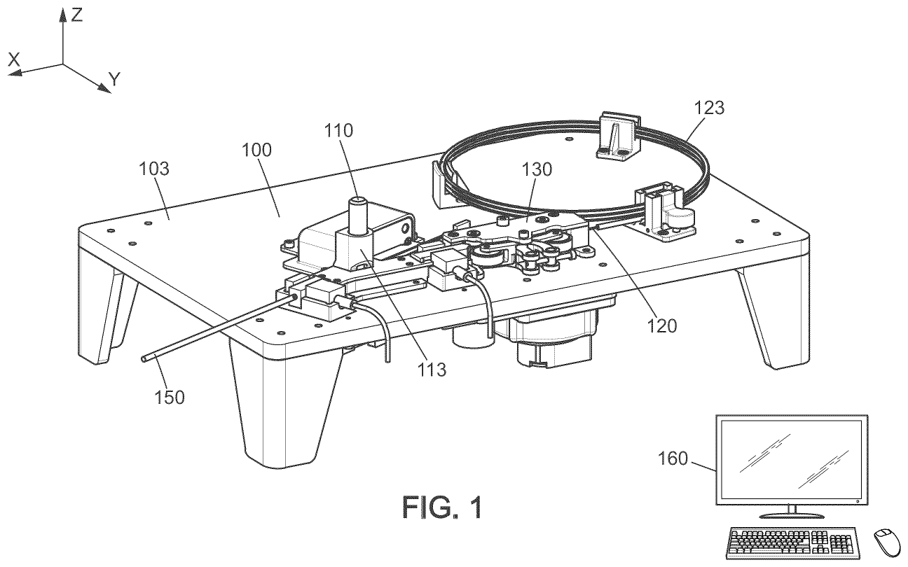

FIG. 1 schematically illustrates a loading and pushing device in one or more embodiment(s).

Referring to FIG. 1, the loading and pushing device 100 may comprise a movable support 103, for example a table positioned proximate to the patient and the medical imaging device, comprising the different portions of the device 100 described hereinafter. For example, the movable support may be located proximate to an imaging system (for example, MRI, scanner, etc.) used to monitor placement of the radioactive seeds in an area to be treated, for example according to brachytherapy.

The device 100 may incorporate a reservoir 110 of radioactive seeds, free with respect to one another, the reservoir 110 may, for example, consist of a removable cartridge fitting into a support 113. In particular, the support 1113 is securely connected to the movable support 103, in particular fastened on the upper surface of a table of the movable support 103.

In order to enable pushing of the radioactive seed located in the loading area towards an area of a patient to be treated, the device 100 may comprise a push cable 120 comprised, at least partially in a protective envelope, for example in the form of a rigid sheath 123. The push cable may be associated with an accumulation system based on a helical shape of the rigid sheath, thereby ensuring a high compactness of the push cable in the retracted position. The rigid helical sheath is connected and secured, at least in some local positions, to the movable support 103, in particular secured to the upper surface of the table forming the movable support 103.

As an example, the push cable may be made of metal such as an alloy of titanium, nickel or stainless steel, or of composite or any other compatible material for operation within an imaging system such as MRI or a scanner, or compatible with imaging systems using magnetic field values comprised between 0 and 5 Teslas.

The movement and guidance of the push cable 120 may be ensured by means of a motor-driven guide and drive device 130 comprised in the device 100 and a control unit 160 allowing controlling the movement of the cable 120. The control unit 160 may be located remotely from the motor-driven guide and drive device and communicate with the device 100 via a telecommunication network (for example, Wi-Fi, Bluetooth, 4G/5G, etc.), or in a wired manner. Alternatively, the control unit 160 is directly integrated into the device 100.

Furthermore, the device 100 may be connected to a catheter 150 (partially shown) configured to allow conveying a pushed radioactive seed, throughout the catheter, by the push cable 120 up to the treatment area (not shown).

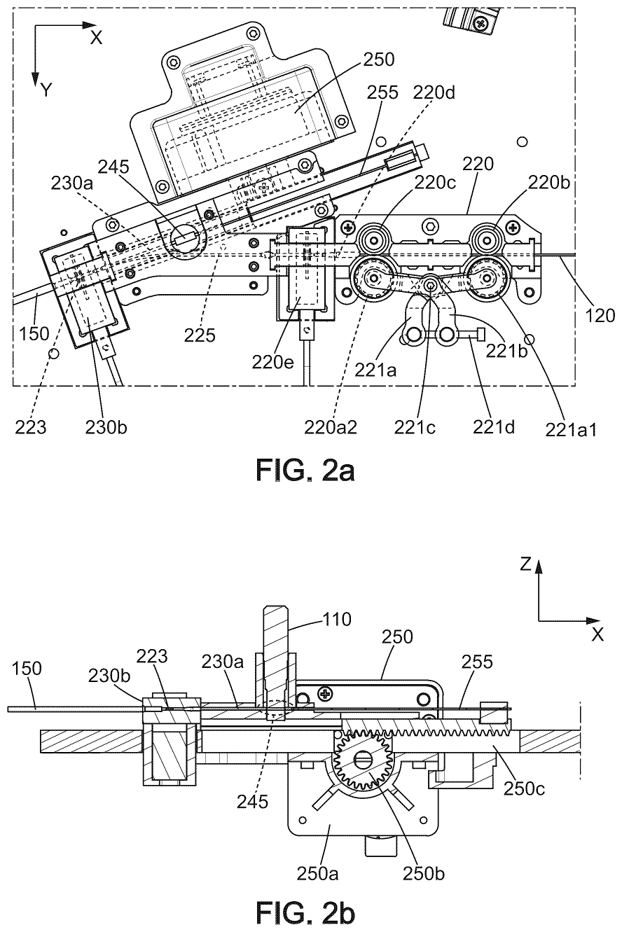

FIGS. 2a and 2b respectively show a partial sectional view of the device 100 according to the planes XY and XZ.

Referring to FIGS. 2a and 2b, the guide and drive device 130 of the push cable 120 may comprise a first actuator 220 allowing setting the cable 120 into movement. This first actuator may comprise a pressing rollers 220a1; 220a2/drive roller 220b pair pinching the push cable, the latter being moved by friction between the rollers 220a1; 220a2/220b driven by a motor (for example, a piezoelectric motor, a servo motor, a stepper motor, etc.) coupled to one of the rollers 220a1; 220a2.

In one or more embodiment(s), the guide and drive device may further comprise a measuring roller 220c matching with one of the pressing rollers 220a1; 220a2, the measuring roller being coupled to a measuring means (for example, an incremental encoder) (not shown) allowing determining a value of the length of the unwound push cable.

The pressure of the pressing rollers 220a1; 220a2 may be configured by means of a pressure adjustment system. The adjustment system may comprise a first support 221a for the first pressing roller 220a1 a and a second support 221b for the second pressing roller 220a2 articulated to each other about an articulation axis 221c, and adjusted by means of a screw tightening system 221d connecting the first support to the second support. The value of the tightening torque exerted on the first and second supports may be within a range of torque value from 0.2 to 3 N·m-1.

Furthermore, to avoid any derailment of the push cable between the rollers, the device 100 may comprise one or more guide(s) 220d in order to safely convey the push cable towards the guide system which comprises a downstream branch 223 forming the loading area for a radioactive seed and an upstream branch 225) which could be defined as a first upstream branch. The upstream branch (or first upstream branch) may be configured so as to extend the downstream branch 223 at a proximal end, and enable the retraction of the push cable in a retracted position thereby clearing the loading area for loading a radioactive seed. The retraction of the push cable is made possible by the use of the accumulation system located downstream of the first upstream branch.

The retracted position of the push cable may be determined by means of a cable detector 220e configured to detect the presence of the end of the push cable in the first upstream branch or in an area located in the first upstream branch.

In order to ensure loading of a radioactive seed from the reservoir 110 towards the downstream branch 223 forming the loading area, the device 100 may comprise a second upstream branch 230a and a seed detector 230b targeting the loading area, the seed detector being configured to detect a presence (for example, a radioactive seed) in the loading area.

Furthermore, the first upstream branch 225 and the second upstream branch 230a are formed by splitting the downstream branch 223 at its proximal end, and the second upstream branch may comprise a pre-loading area 245 into which the radioactive seed reservoir opens via a lateral opening.

In one or more embodiment(s), the downstream branch 223 and the second upstream branch 230a can extend rectilinearly, the downstream branch 223 and the first upstream branch 225 can then form a bend therebetween. For example, the value of the angle of the bend may be within a range of values from 10 to 45 degrees.

During loading of a radioactive seed from the reservoir 110 towards the loading area 223, a radioactive seed is first released in the pre-loading area 245, then conveyed from the pre-loading area towards the loading area via an actuation device comprised in the device 100 and consisting of a second actuator 250 and a push member 255. When the push member is moved by the second actuator, the push member can switch from a retracted initial position, beyond the proximal end of the downstream branch, allowing clearing the pre-loading area 245 for loading a radioactive seed into a deployed position allowing bringing the radioactive seed released from the pre-loading area towards the loading area 223 within the downstream branch.

For example, the push member may be a rectilinear rod made of metal such as an alloy of titanium, nickel or stainless steel, or in composite, or any material suitable for operating within an imaging system such as MRI or the scanner.

The second actuator 250 may consist of a motor 250a as well as a transmission comprising a gear, like for example a gear wheel 250b and a rack 250c which is secured to the push member 255. Thus, driving of the gear wheel by the motor allows advancing or retracting the push member 255.

Furthermore, in general, the different elements (or parts) comprised in the device 100 as described before may be compatible to operate within imaging systems, such as scanners, MRI or imaging systems allowing carrying out echographies. For example, in the case of an imaging system such as MRI, the different elements comprised in the device 100 may be compatible to operate under a magnetic field having Tesla values comprised between 0 and 5 Tesla. Preferably, the different elements comprised in the device 100 may be compatible to operate under a magnetic field having Tesla values comprised between 0 and 3 Tesla.

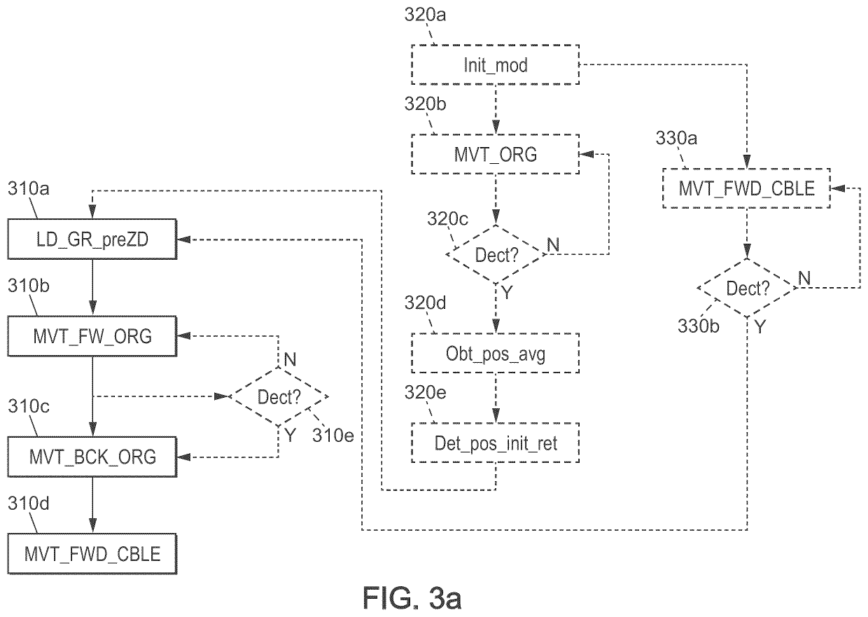

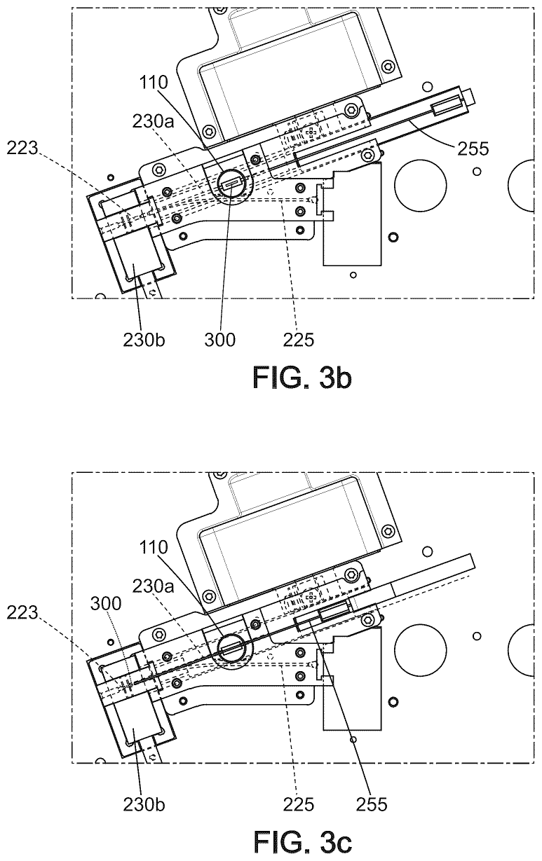

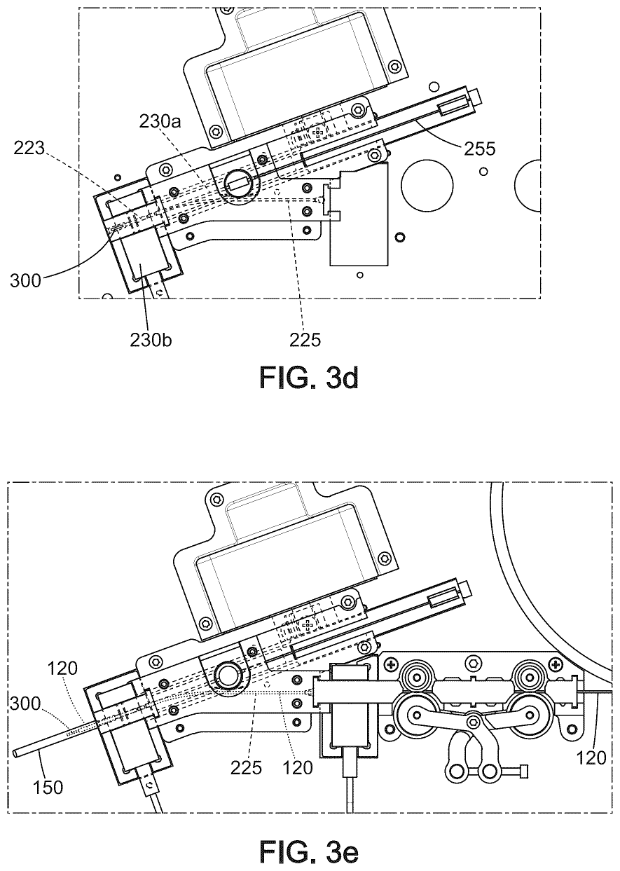

FIGS. 3a to 3e schematically illustrate the method for loading a radioactive seed in the loading area of the device 100.

Upon initialisation of the device 100, the push cable may be in a retracted position and the push member may also be in a retracted initial position so as to clear the different routes, i.e. the upstream branches and the downstream branch, and enable loading of a radioactive seed in the loading area. Hence, no radioactive seed is present in the loading area and the control unit may then receive an input signal of non-detection of radioactive seed by the seed detector targeting the loading area in the downstream branch. A radioactive seed 300 may be released (for example, by gravity or spring by the control unit or manually) 310a in the pre-loading area within in the second upstream branch 230a, and the control unit 160 may then be configured to:
/a/advance 310b the push member 255 by the second actuator so as to push the radioactive seed 300 from the pre-loading area into the second upstream branch 203a up to the loading area in the downstream branch 223.
/b/retract 310c the push member 255 in the second upstream branch 230a by the second actuator so as to clear the proximal end of the downstream branch 223.
/c/advance 310d the push cable 120 by the first actuator so as to push the radioactive seed 300 from the loading area 223 up to a treatment area.

Optionally, and according to one or more embodiment(s), the radioactive seed 300 may be advanced by the push member until detection thereof 310e in the loading area by the seed detector 230b. Once the seed detector detects passage of the seed in the loading area, the radioactive seed may be advanced by the push member up to the beginning of the entrance of the catheter 150.

Optionally, and according to one or more embodiment(s), upon initialisation of the device 100, an initialisation mode 320a may be activated before any loading of a radioactive seed into the pre-loading area so as to determine the retracted initial position of the push member, the control unit may be configured to determine this position (at least in an initialisation mode) by:
/d/controlling 320b the second actuator so as to move the push member 255 until detection 320c of the push member by the seed detector 230b in the loading area,
/e/obtaining 320d an actual position of the push member in the loading area,
/f/determining 320e said retracted initial position of the push member on the basis of the obtained actual position.

For example, the actual position of the push member in the loading area may be determined by one or more round-trip serie(s) within the loading area 223.

Furthermore, like the push member, optionally and in one or more embodiment(s), upon initialisation of the device 100, an initialisation mode may be activated 320a to determine the retracted position of the push cable. This initialisation mode may be the same as that one activated for the determination of the retracted initial position of the push member or may be a different initialisation mode. The control unit may be configured to actuate 330a the first actuator enabling movement of the push cable until detection 330b of its (distal) push end by the cable detector. For example, the control unit 160 advances the cable if the latter is not initially detected, and until detection of its distal end, the signal of the cable detector switching from non-detection into detection when the distal end is in the field of the detector. On the contrary, the control unit retracts the cable if the cable is initially detected until the cable detector 230b no longer detects any cable at a position immediately adjacent to the position of the distal end, the signal of the detector switching from detection into non-detection.

Once the end of the push cable is detected by the cable detector 220e, the push cable 255 can be retracted by a few millimetres (for example, between 2 and 10 millimetres) so as to be placed in the standby position before loading of a seed.

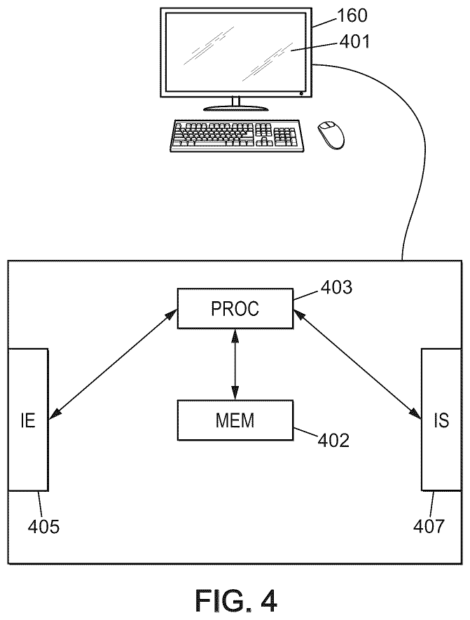

FIG. 4 illustrates a control unit 160 for implementing the method of the present disclosure.

In this embodiment, the control unit 160 may comprise a computer 401, this computer comprising a memory 402 for storing program instructions that can be loaded into a circuit, and able to cause the circuit 403 to execute the method of the present disclosure when the program instructions are managed by the circuit 403.

The memory 402 may also store data and information useful for carrying out the method of the present disclosure as described hereinabove.

For example, the circuit 403 may be:
a processor or a processing unit able to interpret instructions in a computer language, the processor or the processing unit may comprise, be associated with or be related to a memory comprising the instructions, or
the combination of a processor/processing unit and a memory, the processor or the processing unit adapted to interpret instructions in a computer language, the memory comprising said instructions, or
an electronic map in which the sequence of the method is described into silicon, or
a programmable electronic chip such as an FPGA (standing for “Field-Programmable Gate Array”) chip.

This computer may comprise an input interface 405 for receiving input data and an output interface 407 for sending signals, for example control signals. As an example, the input interface may receive input data like a signal of detection of a seed in the loading area sent by the seed detector. For example, the input interface may also receive a detection signal sent by the cable detector. For example, the output interface may send signals so as to control the different actuators of the loading and pushing device.

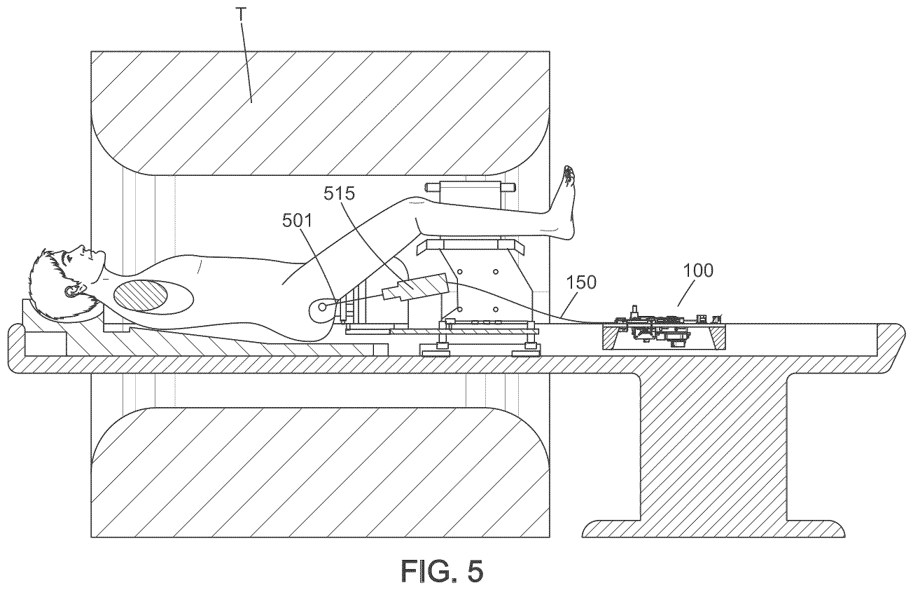

According to another aspect illustrated in FIG. 5, a brachytherapy apparatus is proposed comprising a radioactive seed loading and pushing device 100 according to the present disclosure and a flexible catheter 150 which extends up to a hollow needle 501, the catheter 150 extending a distal end of the downstream branch 223 of the loading device. The push cable is configured to push the radioactive seed from the loading area up to at least one tip of the hollow needle, and in order to deposit the seed in line with the tip in the tissues of the patient.

The radioactive seed loading device 100 may remain fixed, while the hollow needle 501 could be moved, movable, thanks to the flexible catheter which enables freedom of movement between the movable hollow needle 501, and the radioactive seed loading device 100.

This needle is intended to be inserted into the treatment area, for example at the level of the prostate, at one or more target position(s), which are typically determined beforehand in a treatment program. Once the tip of the needle 501, in the desired position, the loading and pushing device is actuated, and so as to deposit the radioactive seed in the position of the tip by pushing of the cable 120.

According to an aspect of the present disclosure, such an insertion of the needle and the depositions of the seeds may be performed by guidance assistance using an imaging device T (for example, enabling real-time operation) like for example magnetic resonance imaging (MRI), as well as a guide robot 515 (or a teleguided arm).

As an example, the guide robot or the teleguided arm may be configured to move and insert the hollow needle at different locations in the area to be treated, so as to deposit radioactive seeds at different positions in the area to be treated (for example, prostate).

According to one aspect of the present disclosure, the guide robot or the teleguided arm may be installed inside or proximate to the imaging device, and may be configured to move according to the three axes X, Y, Z and pivot about each axis.

By proximity, it could be understood a distance comprised between 1 and 2 metre(s) from the imaging device.

Furthermore, the guide robot or the teleguided arm 515 may further be configured to be compatible with imaging devices, i.e. configured to operate within or proximate to (for example, between 1 or 2 metre(s) from) the imaging system such as MRI or a scanner, or within or proximate to (for example, between 1 or 2 metre(s) from) the imaging systems using magnetic field values comprised between 0 and 5 Teslas.

In such a configuration, the hollow needle, the catheter (in part) and the guide robot are internal to the imaging device, while the loading and pushing device is at a distance from the imaging device, in a remote position, and so as to avoid interferences.

Thus, it is possible to deposit radioactive seeds one-by-one in an area to be treated with high accuracy and rapidly, and without it being necessary to move the radioactive seed loading and pushing device, considerably reducing the constraints experienced by the patient in this type of treatment.

According to another aspect, a brachytherapy equipment by magnetic resonance imaging (MRI) guidance is also proposed comprising an MRI imaging scanner comprising a magnet and a magnet tunnel T; as well as a brachytherapy apparatus according to the present disclosure for which the guide robot 515 as well as the hollow needle 501 are positioned internally to the tunnel of the magnet T; while the seed loading and pushing device 100 is external to said tunnel of the magnet.

It should be noted that, in such a configuration, the hollow needle and the catheter (in part) are internal to the tunnel of the magnet, while the loading and pushing device is at a distance from the tunnel, in a remote position, and so as to avoid interferences.

According to another aspect, a method for depositing at least one radioactive seed in a treatment area of a patient to be treated using a brachytherapy treatment apparatus according to the present disclosure or using a brachytherapy treatment equipment according to the present disclosure is also proposed, said patient being installed within said imaging device and said deposition method being imaged in real-time by the imaging device which operates continuously, said method comprising:
/a1/inserting said hollow needle into the tissues of the patient comprised in the treatment area under control of the imaging device,
/b1/loading said at least one radioactive seed into the loading area,
/c1/pushing said at least one radioactive seed from the loading area up to said at least one tip of the hollow needle in order to deposit said at least one seed in line with the tip in the tissues of the patient under control of the imaging device.

The present disclosure finds a particular application for treatments by brachytherapy at low dose rates (LDR) or low radiation rate (LRR), the radioactive seeds implanted by the push cable delivering low radiation over a treatment duration, typically several hours or a few days.

For a high dose rate (HDR) or high radiation rate (HRR) brachytherapy treatment, the push cable of the device 100 according to the present disclosure may be coupled to a radioactive end. This radioactive end is removably coupled to the end of the cable by a fastening means. This cable being deployed by the first actuator, preferably by MRI guidance assistance, and until the radioactive end protrudes from the tip of the hollow needle. The radioactive end is left in the treatment area to deliver a high radiation dose over a short period of time, typically in the range of one or several minute(s).

1-18. (canceled)

19. A brachytherapy treatment apparatus comprising a radioactive seed loading and pushing device, the radioactive seed loading and pushing device comprising:
a seed reservoir containing radioactive seeds, free with respect to one another,
a flexible push cable, and a motor-driven guide and drive device of the push cable configured to make the push cable switch from a retracted position clearing a loading area for a radioactive seed into a deployed position ensuring pushing of the seed from the loading area up to a downstream treatment area, the guide and drive device comprising a first actuator and a guide system for the push cable comprising a downstream branch forming the loading area for the radioactive seed and an upstream branch, extending the downstream branch at a proximal end of the downstream branch, inside which upstream branch a push end of the push cable is configured to retract, in a retracted position of the push cable, and
a system ensuring loading of a seed from the reservoir up to the loading area, the treatment apparatus comprises a flexible catheter which extends up to a hollow needle configured to be inserted into the treatment area, the catheter extending a distal end of the downstream branch of the loading and pushing device and wherein the push cable is configured to push the radioactive seed from the loading area up to the at least one tip of the hollow needle, and in that the upstream branch is a first upstream branch, the system ensuring the loading of a seed from the seed reservoir up to the loading area comprising:
a second upstream branch, the downstream branch extending at its proximal end by splitting into said first upstream branch and said second upstream branch and wherein the seed reservoir opens into the second upstream branch, at a pre-loading area of a seed,
an actuation device comprising a second actuator and a push member configured to switch from a retracted initial position, beyond the proximal end, clearing the pre-loading area by enabling exit of a seed from the seed reservoir into the second branch up to a deployed position for which said push member moves the radioactive seed from the pre-loading area up to the loading area while preventing exit of a seed from the seed reservoir in the pre-loading area,
a control unit configured, starting from the retracted position of the push cable and the retracted initial position of the push member, to:
/a/advance said push member by the second actuator so as to push the radioactive seed from the pre-loading area into the second upstream branch up to the loading area in the downstream branch,
/b/retract said push member into the second upstream branch by the second actuator so as to clear the proximal end of the downstream branch, and
/c/advance the push cable by the first actuator so as to push the radioactive seed from the loading area up to said at least one tip of the hollow needle inserted into the treatment area in order to deposit the seed in line with the tip in the tissues of the patient.

20. The brachytherapy treatment apparatus according to claim 19, wherein the radioactive seed loading and pushing device is fixed, while the hollow needle can be moved, for example movable by means of a robot, thanks to the flexible catheter which allows for a freedom of movement between the movable hollow needle and the fixed radioactive seed loading and pushing device.

21. The brachytherapy treatment apparatus according to claim 19, wherein the seed reservoir is a removable cartridge, and wherein the second upstream branch comprises at the reloading area, a lateral opening (in particular an upper opening) as well as a support configured to ensure fastening of the cartridge, in a removable manner, at the lateral opening.

22. The brachytherapy treatment apparatus according to claim 19, wherein the push member is a rectilinear rod, and wherein:
the downstream branch and the second upstream branch extend rectilinearly,
the downstream branch and the first upstream branch form a bend therebetween.

23. The brachytherapy treatment apparatus according to claim 19, wherein the system ensuring loading of a seed from the seed reservoir up to the loading area comprises a seed detector targeting the loading area configured to detect a presence in the loading area, the seed detector emitting a signal at the input of the control unit, and wherein, in/a/, the control unit is configured to ensure the movement of the seed by the push member from the pre-loading area until detection of the by the seed detector in the loading area.

24. The brachytherapy treatment apparatus according to claim 23, wherein the control unit is configured to determine the retracted initial position of the push member, at least in an initialisation mode, by:
/d/controlling the second actuator so as to move the push member until detection of the push member by the seed detector in the loading area,
/e/obtaining an actual position of the push member in the loading area, and
/f/determining said retracted initial position of the push member on the basis of the obtained actual position.

25. The brachytherapy treatment apparatus according to claim 19, wherein the second actuator comprises a motor, as well as a transmission comprising a gear comprising a gear wheel driven by the motor, and a rack secured to the push member.

26. The brachytherapy treatment apparatus according to claim 19, wherein a cable detector targets a detection area in said first upstream branch and wherein the control unit is configured to determine the retracted position of the push cable, at least in an initialisation mode, for which the first actuator moves the push cable until detection of the push end by the cable detector.

27. The brachytherapy treatment apparatus according to claim 19, wherein the first actuator comprises a pair comprising a motor-driven drive roller and a pressing roller between which the push cable is pinched.

28. The brachytherapy treatment apparatus according to claim 27, comprising a second pair comprising a measuring roller coupled to a measuring means such as an incremental encoder and a second pressing roller between which the push cable is pinched.

29. The brachytherapy treatment apparatus according to claim 28, having a system for adjusting the pressure of the first and second pressing rollers, comprising a first support for the first pressing roller and a second support for the second pressing roller articulated to each other about an articulation axis, as well as a screw tightening system connecting the first support and the second support.

30. The brachytherapy treatment apparatus according to claim 19, wherein the loading device comprises upstream of the first upstream branch a system for accumulating the push cable in the retracted position comprising a rigid sheath for the push cable, the sheath being shaped according to a helicoid.

31. The brachytherapy treatment apparatus according to claim 19, further comprising a guide assistance configured to guide the insertion of the hollow needle into the area to be treated and to guide the deposition of at least one seed, said guide assistance comprising a guide robot configured to be coupled with an imaging device, said guide robot being configured to be placed within or proximate to the imaging device and being further configured to move the hollow needle, and insert the latter into the area to be treated, said imaging device being configured to visualize the movement and the insertion of the hollow needle into the area to be treated, as well as the deposition of the radioactive seed.

32. The brachytherapy treatment apparatus according to claim 31, wherein the guide robot is further configured to be compatible with said imaging device when said imaging device uses magnetic field values comprised between 0 and 5 Teslas.

33. The brachytherapy treatment apparatus according to claim 19, comprising a radioactive head removably coupled to the end of the push cable by a fastening means and wherein the cable is configured to be deployed by the first actuator until the radioactive end protrudes from the tip of the hollow needle.

34. A brachytherapy treatment equipment, comprising the brachytherapy treatment apparatus according to claim 19 and a magnetic resonance imaging device comprising an imaging scanner comprising a magnet and a magnet tunnel (T).

35. The brachytherapy equipment by magnetic resonance imaging guidance according to claim 34, for which the hollow needle is positioned internally to the tunnel of the magnet (T) while the seed loading and pushing device is external to said tunnel of the magnet.

36. A method for depositing at least one radioactive seed in a treatment area of a patient to be treated using the brachytherapy treatment apparatus according to claim 31, said patient being installed within said imaging device and said deposition method being imaged in real-time by the imaging device which operates continuously, said method comprising:
/a1/inserting said hollow needle into the tissues of the patient comprised in the treatment area under control of the imaging device,
/b1/loading said at least one radioactive seed into the loading area,
/c1/pushing said at least one radioactive seed from the loading area up to said at least one tip of the hollow needle in order to deposit said at least one seed in line with the tip in the tissues of the patient under control of the imaging device.

37. A method for depositing at least one radioactive seed in a treatment area of a patient to be treated using the brachytherapy treatment equipment according to of claim 34, said patient being installed within said imaging device and said deposition method being imaged in real-time by the imaging device which operates continuously, said method comprising:
/a1/inserting said hollow needle into the tissues of the patient comprised in the treatment area under control of the imaging device,
/b1/loading said at least one radioactive seed into the loading area,
/c1/pushing said at least one radioactive seed from the loading area up to said at least one tip of the hollow needle in order to deposit said at least one seed in line with the tip in the tissues of the patient under control of the imaging device.
